(19) United States
(12) Patent Application Publication
(10) Pub. No.: US 2025/0197939 A1
(43) Pub. Date: Jun. 19, 2025

(54) Methods and Systems for Measuring Multiple Cell States

(71) Applicants: Washington University, St. Louis, MO (US); The Board of Trustees of the Leland Stanford Junior University, Stanford, CA (US)

(72) Inventors: Aadel Chaudhuri, St. Louis, MO (US); Aaron M. Newman, San Mateo, CA (US)

(73) Assignees: Washington University, St. Louis, MO (US); The Board of Trustees of the Leland Stanford Junior University, Stanford, CA (US)

(21) Appl. No.: 18/848,052

(22) PCT Filed: Mar. 17, 2023
(86) PCT No.: PCT/US2023/064644

§ 371 Date: Sep. 17, 2024

Related U.S. Application Data
(60) Provisional application No. 63/320,927, filed on Mar. 17, 2022.

(51) Int. Cl. C12Q 1/6881; C12Q 1/6886

(52) U.S. Cl. CPC C12Q 1/6881; C12Q 1/6886; C12Q 2600/106; C12Q 2600/154

(57) ABSTRACT

Methods and systems for detecting cell states in a biological sample are disclosed. Methods and systems for predicting a therapeutic response, a severe immune-related adverse event (irAE), a symptomatic irAE, and an irAE grade of a subject to be administered an immunotherapy treatment based on cell states detected from a single biological sample from the subject are also disclosed.

### CROSS-REFERENCE TO RELATED APPLICATIONS

This application claims priority from U.S. Provisional Application Ser. No. 63/320,927 filed on 17 Mar. 2022, which is incorporated herein by reference in its entirety.

### STATEMENT REGARDING FEDERALLY SPONSORED RESEARCH OR DEVELOPMENT

This invention was made with government support under CA238711 and CA142710 awarded by the National Institutes of Health. The government has certain rights in the invention.

### MATERIAL INCORPORATED-BY-REFERENCE

Not applicable.

### FIELD OF THE INVENTION

The present disclosure generally relates to methods for detecting multiple cellular states in bodily fluids or nucleic acid mixtures.

### SUMMARY OF THE INVENTION

Among the various aspects of the present disclosure is the provision of methods and systems for detecting cell states.

In one aspects, a method of determining a cell state composition from a biological sample is disclosed that includes providing the biological sample comprising cell-free DNA, the cell-free DNA comprising a plurality of cell-free DNA fragments; providing a ground-truth reference table comprising a plurality of reference cell and tissue states and associated reference methylation levels;

identifying CpGs within each DNA fragment of the cell-free DNA to determine a methylation level associated with each DNA fragment; comparing the methylation levels of each DNA fragment with the reference methylation levels associated with each cell and tissue state in the ground-truth reference table; assigning each DNA fragment to the cell or tissue state from the ground-truth reference table with an associated reference methylation level that is most similar to the methylation level of the DNA fragment; counting the numbers of DNA fragments assigned to each cell or tissue state of the ground-truth reference table to produce a read-count table; and determining the cell state composition based on the read-count table. In some aspects, the biological sample is a blood sample. In some aspects, the reference methylation values comprise differentially methylated CpGs derived from DNA originating from known cell types and known cell states, optionally of bacterial, viral, fungal, or eukaryotic parasitic origin. In some aspects, the cell-free DNA is plasma-derived. In some aspects, the cell state composition comprises at least two cell types, each cell type comprising at least two cell states. In some aspects, the method further includes inferring a melanoma tumor fraction, a tumor-infiltrating leucocyte fraction, a CD4 TEM level, and any combination thereof based on the cell state composition.

In another aspect, a method of predicting a therapeutic response of a subject to be administered an immunotherapy treatment is disclosed that includes obtaining a biological sample from the subject comprising cell-free DNA, the cell-free DNA comprising a plurality of cell-free DNA fragments; determining the cell state composition of the subject using the method as disclosed herein; inferring a melanoma tumor fraction based on the cell state composition; and predicting the response to the immunotherapy treatment based on the melanoma tumor fraction.

In another aspect, a method of predicting a therapeutic response of a subject to be administered an immunotherapy treatment is disclosed that includes obtaining a biological sample from the subject comprising cell-free DNA, the cell-free DNA comprising a plurality of cell-free DNA fragments; determining the cell state composition of the subject using the method as disclosed herein; inferring a tumor-infiltrating leucocyte fraction based on the cell state composition; and predicting the response to the immunotherapy treatment based on the tumor-infiltrating leukocyte fraction.

In another aspect, a method of predicting a severity of an immune-related adverse event of a subject to be administered an immunotherapy treatment is disclosed that includes obtaining a biological sample from the subject comprising cell-free DNA, the cell-free DNA comprising a plurality of cell-free DNA fragments; determining the cell state composition of the subject using the method as disclosed herein; inferring a CD4 TEM fraction based on the cell state composition; and predicting the severity of the immune-related adverse event based on the CD4 TEM fraction.

In another aspect, a method of predicting a symptomatic immune-related adverse event of a subject to be administered an immunotherapy treatment is disclosed that includes obtaining a biological sample from the subject comprising cell-free DNA, the cell-free DNA comprising a plurality of cell-free DNA fragments; determining the cell state composition of the subject using the method as disclosed herein; inferring a CD4 TEM fraction based on the cell state composition; and predicting the symptomatic irAE based on the CD4 TEM fraction.

In another aspect, a method of predicting a grade of an immune-related adverse event of a subject to be administered an immunotherapy treatment is disclosed that includes obtaining a single biological sample from the subject comprising cell-free DNA, the cell-free DNA comprising a plurality of cell-free DNA fragments; determining the cell state composition of the subject using the method as disclosed herein; inferring a CD4 TEM fraction based on the cell state composition; and predicting the grade of the immune-related adverse event based on the CD4 TEM fraction.

In another aspect, a method of predicting a grade of a therapeutic response, a severe immune-related adverse event (irAE), a symptomatic irAE, an irAE grade, and any combination thereof of a subject to be administered an immunotherapy treatment is disclosed that includes obtaining a sample from the subject comprising cell-free DNA, the cell-free DNA comprising a plurality of cell-free DNA fragments; determining the cell state composition of the subject using the method as disclosed herein; inferring a melanoma tumor fraction, a tumor-infiltrating leucocyte fraction, and a CD4 TEM fraction based on the cell state composition. The method further includes predicting at least one of: the response to the immunotherapy treatment based on at least one of the melanoma tumor fraction and the tumor-infiltrating leucocyte fraction; and the severe immune-related adverse event (irAE), the symptomatic irAE, the irAE grade, and any combination thereof based on the CD4 TEM fraction.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 is a block diagram schematically illustrating a system in accordance with one aspect of the disclosure.

FIG. 2 is a block diagram schematically illustrating a computing device in accordance with one aspect of the disclosure.

FIG. 3 is a block diagram schematically illustrating a remote or user computing device in accordance with one aspect of the disclosure.

FIG. 4 is a block diagram schematically illustrating a server system in accordance with one aspect of the disclosure.

FIG. 5 shows the number of differentially methylated CpGs per purified cell state. Cell states were purified from peripheral blood or tumor tissue, sequenced by genome-wide next-generation methylation sequencing, and then differential methylated region (DMR) analysis was performed bioinformatically. The numbers of differentially methylated CpGs per cell state at different delta thresholds are shown here. Delta depicts the DMR-calling stringency with 0.05 being the least stringent and 0.9 being the most stringent.

FIG. 6A is a graph quantifying melanoma tumor fraction in cell-free DNA from plasma samples from melanoma patients with durable clinical benefit (DCB, response) or no durable benefit (NDB, no response) from immune checkpoint inhibitor (ICI) therapy. Plasma was extracted from pre-treatment blood samples from melanoma patients treated with immune checkpoint blockade. After plasma extraction, cell-free DNA was analyzed for the presence of melanoma tumor signal in plasma cell-free DNA using next-generation methylation sequencing followed by read-counting.

FIG. 6B is a graph of the sensitivity and specificity of the ability to predict the response from the data represented in FIG. 6A.

FIG. 7A is a graph quantifying tumor-infiltrating leukocyte (TIL) fraction in cell-free DNA (ctilDNA fraction) from plasma samples from melanoma patients with durable clinical benefit (DCB, response) or no durable benefit (NDB, no response) from immune checkpoint inhibitor (ICI) therapy. Plasma was extracted from pre-treatment blood samples from melanoma patients treated with immune checkpoint blockade. After plasma extraction, cell-free DNA was analyzed for the presence of TIL signal in plasma cell-free DNA using next-generation methylation sequencing followed by read-counting.

FIG. 7B is a graph of the sensitivity and specificity of the ability to predict the response from the data represented in FIG. 7A.

FIG. 8A is a graph quantifying CD4 T effector memory (TEM) fraction from cell-free DNA from plasma samples from melanoma patients with no severe immune-related adverse effects (irAE) or with severe irAE from immune checkpoint inhibitor (ICI) immunotherapy. Plasma was extracted from pre-treatment blood samples from melanoma patients treated with immune checkpoint blockade. After plasma extraction, cell-free DNA was analyzed for the presence of CD4 TEM signal in plasma cell-free DNA using next-generation methylation sequencing followed by read-counting.

FIG. 8B is a graph of the sensitivity and specificity of the ability to predict severe immune-related adverse events from the data represented in FIG. 8A.

FIG. 9A is a graph quantifying CD4 T effector memory (TEM) cell levels in cell-free DNA from plasma samples from melanoma patients with no symptomatic immune-related adverse effects (irAE) or with symptomatic irAE from immune checkpoint inhibitor (ICI) immunotherapy.

FIG. 9B is a graph of the sensitivity and specificity of the ability to predict symptomatic immune-related adverse events from the data represented in FIG. 9A.

FIG. 10 is a graph quantifying CD4 T effector memory (TEM) cell fraction in cell-free DNA plasma samples from melanoma patients on an immune-related adverse effect (irAE) grade-by-grade basis (0-4), showing that the described method can predict irAE on a grade by grade basis. Plasma was extracted from pre-treatment blood samples from melanoma patients treated with immune checkpoint blockade. After plasma extraction, cell-free DNA was analyzed for the presence of CD4 TEM signal in plasma cell-free DNA using next-generation methylation sequencing followed by read-counting, which was correlated clinically with the immune-related adverse event (irAE) severity (irAE grade measured by CTCAE v5).

FIG. 11 is a plot showing the expression of various cell states and types in regard to the severity of irAE present in a patient, showing that CD4 TEMs are most significantly associated with severe irAE compared to other cell states and types.

### DETAILED DESCRIPTION OF THE INVENTION

The present disclosure is based, at least in part, on the discovery that cell states can be measured in a tissue or bodily fluid. It is noted that the scope of the method is not limited to DNA methylation or plasma-derived cell-free DNA. It can be applied to any sequenced nucleic acid mixture (i.e., DNA or RNA) from any cellular or cell-free DNA source (i.e., any bodily fluid or tissue source). Although examples disclosed here use bisulfite/methylation sequencing, this method can be used with any type of next-generation sequencing or microarray technology known in the art (see e.g., Rajesh et al. 2017—Next-Generation Sequencing Methods; Current Developments in Biotechnology and Bioengineering: Functional Genomics and Metabolic Engineering 2017, Pages 143-158; Moss et al. 2018 Comprehensive human cell-type methylation atlas reveals origins of circulating cell-free DNA in health and disease. Nat Commun 9, 5068; Bumgarner, 2013, Overview of DNA Microarrays: Types, Applications, and Their Future, Volume101, Issue 1 Pages 22.1.1-22.1.11, for example).

As shown herein, the presently disclosed method enables the detection and profiling of a tumor microenvironment (including tumor-infiltrating leukocytes and tumor cell states) using a blood-based liquid biopsy approach. This is performed through methylation sequencing of plasma-derived cell-free DNA. Individual single-cell states are profiled from bulk using either genome-wide or targeted bisulfite sequencing (e.g., leukocyte and tumor cell states by counting or, optionally, deconvolving plasma methylation sequencing data).

This method is based on single-molecule counting, which allows one to enumerate and classify molecules (DNA or RNA reads) into reference bins on a molecule-by-molecule level. As such, the method involves counting. It starts with individual molecules, and by enumerating and classifying them one by one, the method is able to learn how the full system is comprised molecule by molecule. This can make this method high resolution compared to alternative methods.

In some embodiments, a machine learning model may be used to enumerate and classify DNA or RNA molecules into reference bins. In these embodiments, the machine learning model may be trained using DNA or RNA molecules obtained from isolated cell types or cell states as described herein. Any machine learning architecture may be used to implement the methods disclosed herein including, but not limited to, random forest, support vector machine, logistic regression, KNN, and K-means. In some aspects, gradient-boosted algorithms and AdaBoosted algorithms can also be applied to further optimize the read-counting algorithm as implemented using machine learning systems and methods.

On the other hand, deconvolution starts by looking at the entire bulk sequenced mixture as a whole, then optimally tries to weigh and add cell-type-specific signatures together in order to achieve the mixture-representing matrix. Thus the deconvolution method has intrinsically much lower resolution and is fundamentally different from the disclosed method.

In various aspects, a read-counting method for deconvolving cell-free DNA methylation is disclosed that provides for the determination of multiple cell states (≥2). The read-counting method provides exquisite granularity with the ability to distinguish and quantify several cell states (even related ones) from one another. The disclosed read-counting method can be used to noninvasively profile the tumor and the tumor microenvironment from a body fluid sample, and the cell states identified using the read-counting method can be used to noninvasively predict treatment response via “liquid biopsy”.

In some aspects, the read-counting method includes identifying CpGs on a per-fragment level in cell-free DNA. The term “CpG”, as used herein, refers to the nucleotide sequence cytosine-guanine (CG) at which methylation commonly occurs.

In some aspects, methylation levels, defined herein as the portion of the CpG sites that are actively methylated, are measured. In various aspects, methylation levels may be measured using any suitable method including, but not limited to, bisulfite/methylation sequencing.

In various aspects, methylation levels of the CpGs can be compared to ground-truth reference tables of known cell/tissue states, and the CpG sites per fragment can be collated to assign the cell-free DNA fragment to a cell state within the reference tables. In some aspects, each ground-truth reference table is obtained by analyzing cell-free DNA samples obtained from sources with a known single cell type and/or single cell state, including, but not limited to, the various cells and cell states described herein. Cell-free DNA fragments are counted until all fragments have been assessed and assigned to a ground-truth reference table. In some embodiments, the results can be optimized further using machine learning. The patterns of assignment of cell-free DNA fragments from the cell-free DNA mixture can be used to determine the cell-free DNA mixture's cell state composition. In some embodiments, methylation levels can be represented by the number of CpG sites. In some embodiments, methylation levels can be represented as the portion of the CpG sites that are actively methylated as measured by bisulfate/methylation sequencing.

It is specifically shown that immunotherapy toxicity (and treatment toxicity more generally) can be predicted from cell-free DNA methylation cell state analysis. Peripheral blood cell states can be granularly profiled using the method, and activated CD4 T effector memory cells can be quantified from cell-free DNA, which enables pre-treatment and early on-treatment prediction of immunotherapy toxicity (i.e., immune-related adverse events). Moreover, the method can concurrently predict both treatment response and toxicity using the same assay. Additionally, the assay can be applied to concurrently quantify a wide range of cell states that comprehensively represent human health and disease, essentially an atlas (i.e., by measuring multiple cells, tissues, and microbial types/states that either we have sequenced or that are present in public/published methylation datasets), to predict and monitor risk for a wide range of physiologic states, disorders, infections, and diseases.

This method can enumerate and distinguish cell types and/or cellular states without the need for solid tissue biopsies. “Cellular states” can be defined as context-dependent versions of a given cell type (e.g., normal vs. tumor-associated CD8 T cells). This unique capability allows the presently disclosed noninvasive approach to measure the non-malignant cells within a tumor and distinguish them from their normal tissue counterparts. It is presently believed that this is the first time this has been accomplished. Previous studies have exclusively focused on distinguishing cell types, tissue types, and cancer vs. normal cells—all of these classifications are less granular than cellular states.

The disclosed method is dependent on prior knowledge of cell state-specific signatures (e.g., from known cells). These signatures allow this approach to enumerate specific cell types and cellular states directly from methylation signals in cell-free DNA. Such signatures can be derived by physically isolating cell states of interest by FACS or by inferring them via single-cell bisulfite sequencing. However, these methods have major shortcomings, including the variable loss of specific cell types by tissue dissociation, the sensitivity, and specificity of the antibody panel (needed for FACS), the low amounts of tissue typically obtained from tumor biopsies, etc. Therefore, a novel alternative has been developed to complement these techniques. The approach is based on inferring cell state signatures directly from bulk tumor methylation profiles. This can be done via statistical deconvolution in a process that is essentially the inverse of measuring cell composition from bulk methylation profiles (e.g., CIBERSORTx; Newman et al. (2019) Nature Biotechnology (37) 773-782). This novel approach can be used to flexibly generate signatures for nearly any cellular state of interest without antibodies, living cells, or physical cell isolation.

The read-counting method enables high-resolution methylation cell state analysis of plasma cell-free DNA, which has been used in the present disclosure to identify 24 distinct cell states in blood plasma. As described herein, the method is able to concurrently predict immunotherapy response and toxicity from the same plasma sample and sequencing result. In some embodiments, this immunotherapy response and toxicity prediction can be performed pre-treatment. These methods also enable the use of these methods in clinical settings.

It is noted that the scope of the method is not limited to DNA methylation or plasma-derived cell-free DNA. It can be applied to any sequenced nucleic acid mixture from any cellular or cell-free DNA or RNA source (i.e., any bodily fluid or tissue source).

### Methods and Systems for Noninvasively Measuring Cell States in Bodily Fluids

The present disclosure provides for the noninvasive measurement of measuring cell states in bodily or biological fluids. More specifically, the enumeration of specific cell types and cellular states directly from methylation signals present in cell-free DNA.

As described herein, this technology is capable of identifying a cell type and a cell state in a single cell or a bulk mixture of cells. A cell state can be defined as the phenotype of a cell. The phenotype of a cell can be a ‘homeostatic phenotype’ implying plasticity resulting from a dynamically changing yet characteristic pattern of gene/protein expression.

The methods described herein can be applied to many commercial/biomedical problems, including immunotherapy response assessment, immunotherapy toxicity assessment, response of any tumor to any drug, tracking the tumor microenvironment noninvasively in research, clinical, or commercial applications, and enabling a true liquid biopsy of the tumor that includes both cancer and tumor microenvironment profiling.

This technology can be used in a broad variety of applications using any type of epigenetics data (i.e., whole-genome bisulfite sequencing, reduced representation bisulfite sequencing, methylation microarrays, etc.) on any bodily fluid (e.g., urine, saliva, plasma, stool, etc.).

This method enables the detection and profiling of the tumor microenvironment (including tumor-infiltrating leukocytes and tumor cell states) using a liquid biopsy approach. We do this through methylation sequencing of plasma-derived cell-free DNA, followed by digital cytometry (deconvolution). We profiled individual single-cell states from bulk using either genome-wide or targeted bisulfite sequencing (e.g., leukocyte and tumor cell states by deconvolving plasma methylation sequencing data).

Although this method is shown here for detecting cell states and cell types in cell-free DNA, it can also be a useful method for use with nucleic acid sequencing of any length. The nucleic acid can be full-length DNA, a DNA fragment, cell-free DNA, RNA, or cell-free nucleic acid fragment assigned to a cell type originating from a tumor cell, an infected cell, a damaged cell, a normal cell, a bacterial cell, an organ or tissue cell, a tissue cell that secretes cfDNA, microbes such as bacteria, viruses (DNA or RNA), fungi, or eukaryotic parasites, for example. In some embodiments, the DNA fragment can be about 300 base pairs or less. It is also noted that the scope of the method is not limited to DNA methylation or plasma-derived cell-free DNA. It can be applied to any sequenced or microarray-profiled nucleic acid mixture from any cellular or cell-free DNA source (i.e., any bodily fluid or tissue source).

As described herein, one or more CpG methylation sites are detected. The CpG methylation sites can be co-associated (e.g., proximal or nearby to each other) between any number of base pairs along the length of a DNA molecule. In some embodiments, the number of base pairs between co-associated CpGs can be between about 1 base pair (bp) and about 1000 bps (proximal or nearby to each other), between 1 bp and about 500 bps, or between about 1 bp and about 300 bps. For example, the nearby or proximal CpGs can be separated by about 1 bp; about 2 bps; about 3 bps; about 4 bps; about 5 bps; about 6 bps; about 7 bps; about 8 bps; about 9 bps; about 10 bps; about 11 bps; about 12 bps; about 13 bps; about 14 bps; about 15 bps; about 16 bps; about 17 bps; about 18 bps; about 19 bps; about 20 bps; about 21 bps; about 22 bps; about 23 bps; about 24 bps; about 25 bps; about 26 bps; about 27 bps; about 28 bps; about 29 bps; about 30 bps; about 31 bps; about 32 bps; about 33 bps; about 34 bps; about 35 bps; about 36 bps; about 37 bps; about 38 bps; about 39 bps; about 40 bps; about 41 bps; about 42 bps; about 43 bps; about 44 bps; about 45 bps; about 46 bps; about 47 bps; about 48 bps; about 49 bps; about 50 bps; about 51 bps; about 52 bps; about 53 bps; about 54 bps; about 55 bps; about 56 bps; about 57 bps; about 58 bps; about 59 bps; about 60 bps; about 61 bps; about 62 bps; about 63 bps; about 64 bps; about 65 bps; about 66 bps; about 67 bps; about 68 bps; about 69 bps; about 70 bps; about 71 bps; about 72 bps; about 73 bps; about 74 bps; about 75 bps; about 76bps; about 77 bps; about 78 bps; about 79 bps; about 80 bps; about 81 bps; about 82 bps; about 83 bps; about 84 bps; about 85 bps; about 86 bps; about 87 bps; about 88 bps; about 89 bps; about 90 bps; about 91 bps; about 92 bps; about 93 bps; about 94 bps; about 95 bps; about 96 bps; about 97 bps; about 98 bps; about 99 bps; about 100 bps; about 101 bps; about 102 bps; about 103 bps; about 104 bps; about 105 bps; about 106 bps; about 107 bps; about 108 bps; about 109 bps; about 110 bps; about 111 bps; about 112 bps; about 113 bps; about 114 bps; about 115 bps; about 116 bps; about 117 bps; about 118 bps; about 119 bps; about 120bps; about 121 bps; about 122 bps; about 123 bps; about 124 bps; about 125 bps; about 126 bps; about 127 bps; about 128 bps; about 129 bps; about 130 bps; about 131 bps; about 132 bps; about 133 bps; about 134 bps; about 135 bps; about 136 bps; about 137 bps; about 138 bps; about 139 bps; about 140 bps; about 141 bps; about 142 bps; about 143 bps; about 144 bps; about 145 bps; about 146 bps; about 147 bps; about 148 bps; about 149 bps; about 150 bps; about 151 bps; about 152 bps; about 153 bps; about 154 bps; about 155 bps; about 156 bps; about 157 bps; about 158 bps; about 159 bps; about 160 bps; about 161 bps; about 162 bps; about 163 bps; about 164 bps; about 165 bps; about 166 bps; about 167 bps; about 168 bps; about 169 bps; about 170 bps; about 171 bps; about 172 bps; about 173 bps; about 174 bps; about 175 bps; about 176 bps; about 177 bps; about 178 bps; about 179 bps; about 180 bps; about 181 bps; about 182 bps; about 183 bps; about 184 bps; about 185 bps; about 186 bps; about 187 bps; about 188 bps; about 189 bps; about 190 bps; about 191 bps; about 192 bps; about 193 bps; about 194 bps; about 195 bps; about 196 bps; about 197 bps; about 198 bps; about 199 bps; about 200 bps; about 201 bps; about 102 bps; about 203 bps; about 204 bps; about 205 bps; about 206 bps; about 207 bps; about 208 bps; about 209 bps; about 210 bps; about 211 bps; about 212 bps; about 213 bps; about 214 bps; about 215 bps; about 216 bps; about 217 bps; about 218 bps; about 219 bps; about 220 bps; about 221 bps; about 222 bps; about 223 bps; about 224 bps; about 225 bps; about 226 bps; about 227 bps; about 228 bps; about 229 bps; about 230 bps; about 231 bps; about 232 bps; about 233 bps; about 234 bps; about 235 bps; about 236 bps; about 237 bps; about 238 bps; about 239 bps; about 240 bps; about 241 bps; about 242 bps; about 243 bps; about 244 bps; about 245 bps; about 246 bps; about 247 bps; about 248 bps; about 249 bps; about 250 bps; about 251 bps; about 252 bps; about 253 bps; about 254 bps; about 255 bps; about 256 bps; about 257 bps; about 258 bps; about 259 bps; about 260 bps; about 261 bps; about 262 bps; about 263 bps; about 264 bps; about 265 bps; about 266 bps; about 267 bps; about 268 bps; about 269 bps; about 270 bps; about 271 bps; about 272 bps; about 273 bps; about 274 bps; about 275 bps; about 276 bps; about 277 bps; about 278 bps; about 279 bps; about 280 bps; about 281 bps; about 282 bps; about 283 bps; about 284 bps; about 285 bps; about 286 bps; about 287 bps; about 288 bps; about 289 bps; about 290 bps; about 291 bps; about 292 bps; about 293 bps; about 294 bps; about 295 bps; about 296 bps; about 297 bps; about 298 bps; about 299 bps; or about 300 bps.

A control sample or a reference sample as described herein can be a sample from a healthy subject. A reference value can be used in place of a control or reference sample, which was previously obtained from a healthy subject or a group of healthy subjects. A control sample or a reference sample can also be a sample with a known cellular or tumor composition.

### Computing Systems and Devices

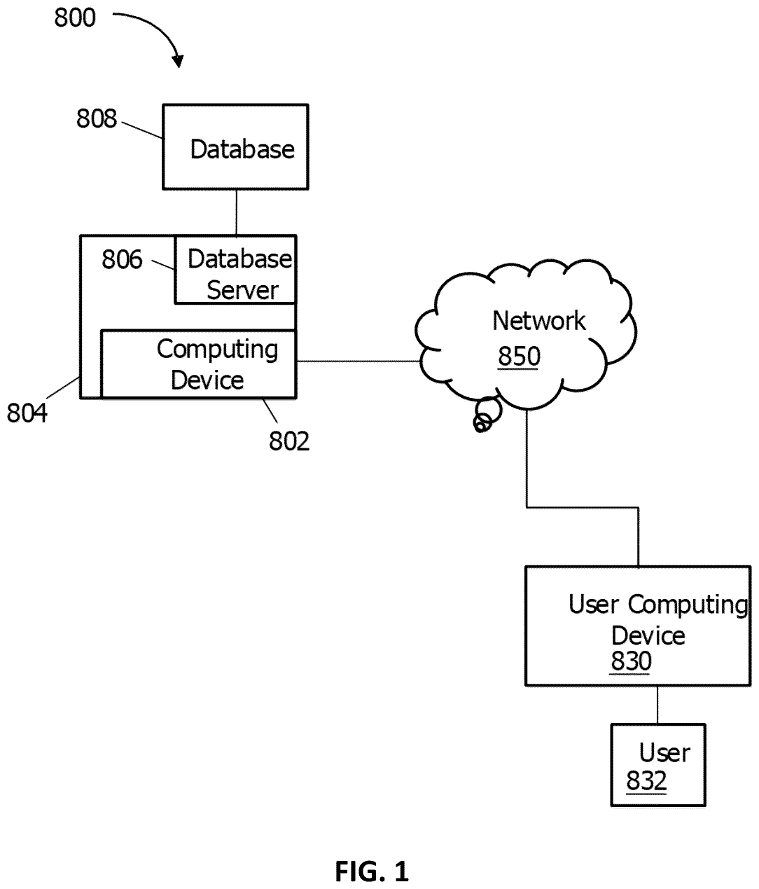

In various aspects, the methods described herein are implemented using computing devices and systems. FIG. 1 depicts a simplified block diagram of a system 800 for implementing the methods described herein. As illustrated in FIG. 1, the system 800 may be configured to implement at least a portion of the tasks associated with the disclosed method. The system 800 may include a computing device 802. In one aspect, the computing device 802 is part of a server system 804, which also includes a database server 806. The computing device 802 is in communication with a database 808 through the database server 806 via a network. The network 850 may be any network that allows local area or wide area communication between the devices. For example, the network 850 may allow communicative coupling to the Internet through at least one of many interfaces including, but not limited to, at least one of a network, such as the Internet, a local area network (LAN), a wide area network (WAN), an integrated services digital network (ISDN), a dial-up-connection, a digital subscriber line (DSL), a cellular phone connection, and a cable modem. The user computing device 830 may be any device capable of accessing the Internet including, but not limited to, a desktop computer, a laptop computer, a personal digital assistant (PDA), a cellular phone, a smartphone, a tablet, a phablet, wearable electronics, smartwatch, or other web-based connectable equipment or mobile devices.

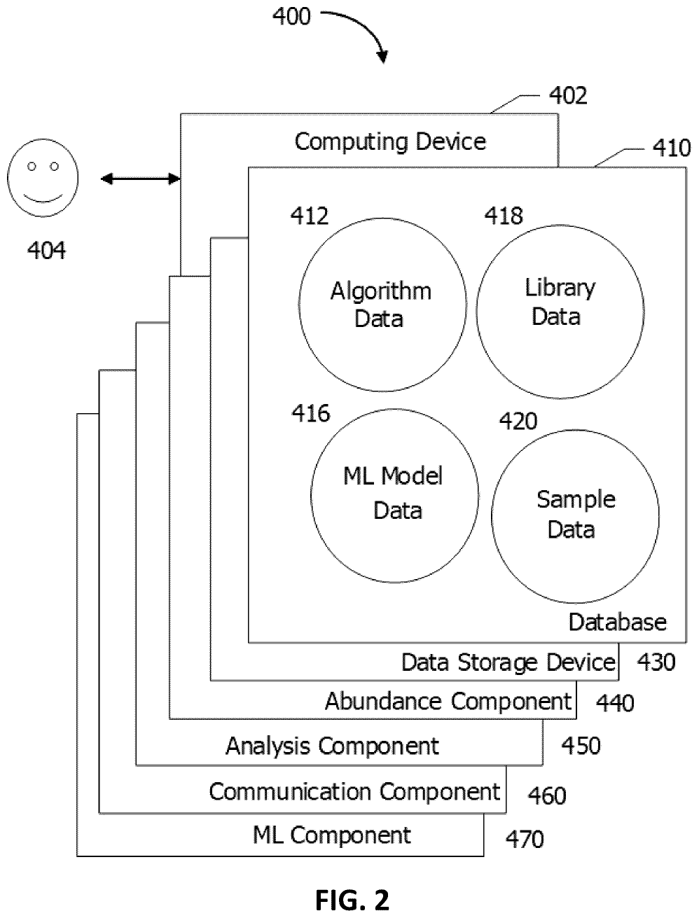

In other aspects, the computing device 802 is configured to perform a plurality of tasks associated with the method of detecting abundances of cell states and/or cell types as described herein. FIG. 2 depicts a component configuration 400 of a computing device 402, which includes a database 410 along with other related computing components. In some aspects, the computing device 402 is similar to computing device 802 (shown in FIG. 1). A user 404 may access components of the computing device 402. In some aspects, the database 420 is similar to the database 808 (shown in FIG. 1).

In one aspect, the database 410 includes library data 418, algorithm data 412, ML model data 416, and sample data 420. In one aspect, the library data 418 includes entries of a library defining characteristics of different cell types or cell states for which the abundance is detected as described herein. Non-limiting examples of library data 418 include entries of a CpG library, entries of a methylation haplotype block (MHB) library, and a signature matrix. As used herein, a CpG library is defined as a plurality of entries in which each entry includes a differentially methylated CpG site indicative of one of the cell types or cell states. In some aspects, the differentially methylated CpG sites are additionally co-associated

CpG sites. As used herein, a co-associated CpG site refers to a differentially methylated CpG site characterizing one of the cell types or cell states that is positioned at a distance of no more than about 200 bp from an additional differentially methylated CpG site characterizing the same cell type or cell state. As used herein, an MHB library is defined as a plurality of entries in which each entry includes at least two co-associated CpG sites indicative of one of the cell types or cell states. As used herein, a signature matrix comprises a plurality of differentially methylated CpG sites characterizing all of the at least one cell type or cell state. The signature matrix is used as part of a digital deconvolution method as described herein. Non-limiting examples of suitable digital deconvolution methods include CIBERSORTx.

In various aspects, algorithm data 412 includes any parameters used to implement the methods as described herein. Non-limiting examples of suitable algorithm data 412 include any values of parameters defining the calculation of abundance counts, relative abundances, absolute abundances, and any other relevant parameter. Non-limiting examples of ML model data 416 include any values of parameters defining the machine learning models used to optimize CpG libraries, to perform digital deconvolution, and any other transformation, classification, or other task in accordance with the methods described herein. Non-limiting examples of sample data 420 include any plurality of reads associated with the biological sample analysis in accordance with the methods described herein, including DNA sequences, RNA sequences, DNA methylation sequences, and any other suitable nucleic acid sequence.

The computing device 402 also includes a number of components that perform specific tasks. In the exemplary aspect, the computing device 402 includes a data storage device 430, an abundance component 440, an analysis component 450, an ML component 470, and a communication component 460. The data storage device 430 is configured to store data received or generated by the computing device 402, such as any of the data stored in database 410 or any outputs of processes implemented by any component of the computing device 402. The abundance component 450 is configured to transform the plurality of reads associated with a sample into at least one abundance, at least one relative abundance, at least any absolute abundance, or any combination thereof for each of the at least one cell type or cell state to be detected in accordance with the methods described herein. The analysis component 450 is configured to perform any additional analysis of any of the abundances produced in association with the methods described. Non-limiting examples of additional analyses performed using the analysis component 450 include diagnosis of a disease or disorder such as cancer or sepsis, classification of a patient into a category such as a responder or non-responder to a treatment, determination of a treatment efficacy, and any other suitable analysis. In various aspects, the ML component 470 is configured to implement any of the machine learning model-based transformations and analyses as described herein. Non-limiting examples of transformations or analyses implemented using the ML component 470 include digital deconvolution of the cell types or cell states based on a plurality of reads in a mixed sample. Optimization of a CpG library or an MHB library, or any other suitable transformation or analysis is in accordance with the methods described herein.

The communication component 460 is configured to enable communications of the computing device 402 over a network, such as network 850 (shown in FIG. 1), or a plurality of network connections using predefined network protocols such as TCP/IP (Transmission Control Protocol/Internet Protocol).

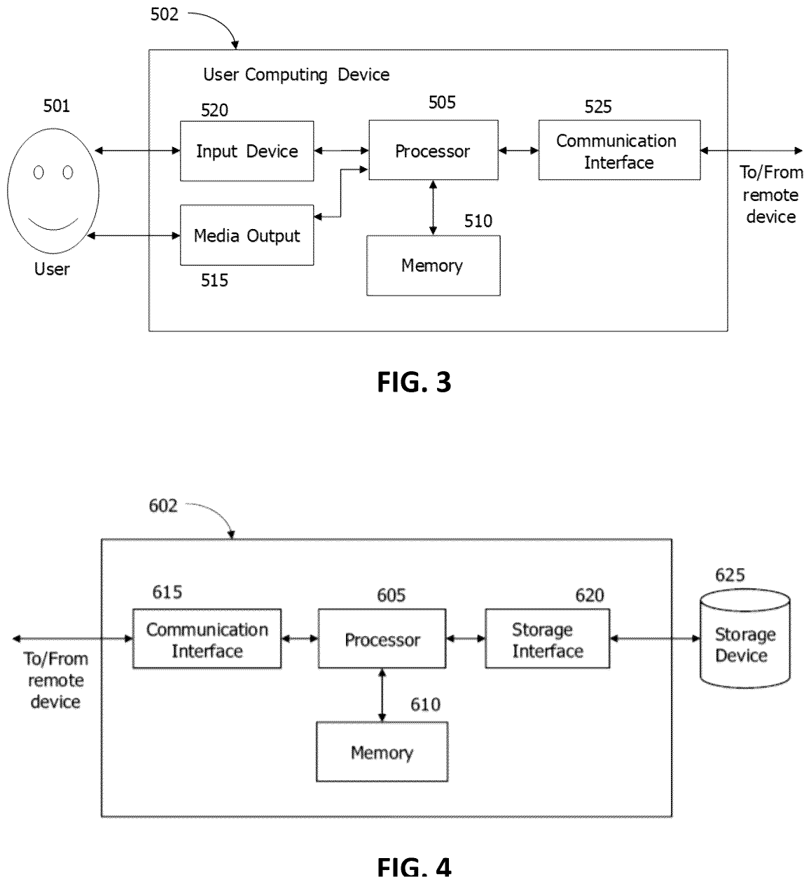

FIG. 3 depicts a configuration of a remote or user computing device 502, such as the user computing device 830 (shown in FIG. 1). The computing device 502 may include a processor 505 for executing instructions. In some aspects, executable instructions may be stored in a memory area 510. Processor 505 may include one or more processing units (e.g., in a multi-core configuration). Memory area 510 may be any device allowing information such as executable instructions and/or other data to be stored and retrieved. Memory area 510 may include one or more computer-readable media.

Computing device 502 may also include at least one media output component 515 for presenting information to a user 501. Media output component 515 may be any component capable of conveying information to user 501. In some aspects, media output component 515 may include an output adapter, such as a video adapter and/or an audio adapter. An output adapter may be operatively coupled to processor 505 and operatively coupleable to an output device such as a display device (e.g., a liquid crystal display (LCD), organic light-emitting diode

(OLED) display, cathode ray tube (CRT), or “electronic ink” display) or an audio output device (e.g., a speaker or headphones). In some aspects, media output component 515 may be configured to present an interactive user interface (e.g., a web browser or client application) to user 501.

In some aspects, computing device 502 may include an input device 520 for receiving input from user 501. Input device 520 may include, for example, a keyboard, a pointing device, a mouse, a stylus, a touch-sensitive panel (e.g., a touchpad or a touch screen), a camera, a gyroscope, an accelerometer, a position detector, and/or an audio input device. A single component such as a touch screen may function as both an output device of media output component 515 and input device 520.

Computing device 502 may also include a communication interface 525, which may be communicatively coupleable to a remote device. Communication interface 525 may include, for example, a wired or wireless network adapter or a wireless data transceiver for use with a mobile phone network (e.g., Global System for Mobile communications (GSM), 3G, 4G, or Bluetooth) or other mobile data network (e.g., Worldwide Interoperability for Microwave Access (WIMAX)).

Stored in memory area 510 are, for example, computer-readable instructions for providing a user interface to user 501 via media output component 515 and, optionally, receiving and processing input from input device 520. A user interface may include, among other possibilities, a web browser, and client application. Web browsers enable users 501 to display and interact with media and other information typically embedded on a web page or a website from a web server. A client application allows users 501 to interact with a server application associated with, for example, a vendor or business.

FIG. 4 illustrates an example configuration of a server system 602. Server system 602 may include, but is not limited to, database server 806 and computing device 802 (both shown in FIG. 1). In some aspects, server system 602 is similar to server system 804 (shown in FIG. 1). Server system 602 may include a processor 605 for executing instructions. Instructions may be stored in a memory area 625, for example. Processor 605 may include one or more processing units (e.g., in a multi-core configuration).

Processor 605 may be operatively coupled to a communication interface 615 such that server system 602 may be capable of communicating with a remote device such as user computing device 830 (shown in FIG. 1) or another server system 602. For example, communication interface 615 may receive requests from user computing device 830 via a network 850 (shown in FIG. 1).

Processor 605 may also be operatively coupled to a storage device 625. Storage device 625 may be any computer-operated hardware suitable for storing and/or retrieving data. In some aspects, storage device 625 may be integrated into server system 602. For example, server system 602 may include one or more hard disk drives as storage device 625. In other aspects, storage device 625 may be external to server system 602 and may be accessed by a plurality of server systems 602. For example, storage device 625 may include multiple storage units such as hard disks or solid-state disks in a redundant array of inexpensive disks (RAID) configuration. Storage device 625 may include a storage area network (SAN) and/or a network-attached storage (NAS) system.

In some aspects, processor 605 may be operatively coupled to storage device 625 via a storage interface 620. Storage interface 620 may be any component capable of providing processor 605 with access to storage device 625.

Storage interface 620 may include, for example, an Advanced Technology Attachment (ATA) adapter, a Serial ATA (SATA) adapter, a Small Computer System Interface (SCSI) adapter, a RAID controller, a SAN adapter, a network adapter, and/or any component providing processor 605 with access to storage device 625.

Memory areas 510 (shown in FIGS. 3) and 610 may include, but are not limited to, random access memory (RAM) such as dynamic RAM (DRAM) or static RAM (SRAM), read-only memory (ROM), erasable programmable read-only memory (EPROM), electrically erasable programmable read-only memory (EEPROM), and non-volatile RAM (NVRAM). The above memory types are examples only, and are thus not limiting as to the types of memory usable for the storage of a computer program.

The computer systems and computer-implemented methods discussed herein may include additional, less, or alternate actions and/or functionalities, including those discussed elsewhere herein. The computer systems may include or be implemented via computer-executable instructions stored on non-transitory computer-readable media. The methods may be implemented via one or more local, remote, o cloud-based processors, transceivers, servers, and/or sensors (such as processors, transceivers, servers, and/or sensors mounted on vehicle or mobile devices, or associated with smart infrastructure or remote servers), and/or via computer-executable instructions stored on non-transitory computer-readable media or medium.

In some aspects, a computing device is configured to implement machine learning, such that the computing device “learns” to analyze, organize, and/or process data without being explicitly programmed. Machine learning may be implemented through machine learning (ML) methods and algorithms. In one aspect, a machine learning (ML) module is configured to implement ML methods and algorithms. In some aspects, ML methods and algorithms are applied to data inputs and generate machine learning (ML) outputs. Data inputs may include but are not limited to: images or frames of a video, object characteristics, and object categorizations. Data inputs may further include: sensor data, image data, video data, telematics data, authentication data, authorization data, security data, mobile device data, geolocation information, transaction data, personal identification data, financial data, usage data, weather pattern data, “big data” sets, and/or user preference data. ML outputs may include but are not limited to: a tracked shape output, categorization of an object, categorization of a type of motion, a diagnosis based on the motion of an object, motion analysis of an object, and trained model parameters ML outputs may further include: speech recognition, image or video recognition, functional connectivity data, medical diagnoses, statistical or financial models, autonomous vehicle decision-making models, robotics behavior modeling, fraud detection analysis, user recommendations and personalization, game Al, skill acquisition, targeted marketing, big data visualization, weather forecasting, and/or information extracted about a computer device, a user, a home, a vehicle, or a part of a transaction. In some aspects, data inputs may include certain ML outputs.

In some aspects, at least one of a plurality of ML methods and algorithms may be applied, which may include but are not limited to: linear or logistic regression, instance-based algorithms, regularization algorithms, decision trees, Bayesian networks, cluster analysis, association rule learning, artificial neural networks, deep learning, dimensionality reduction, and support vector machines. In various aspects, the implemented ML methods and algorithms are directed toward at least one of a plurality of categorizations of machine learning, such as supervised learning, unsupervised learning, and reinforcement learning.

In one aspect, ML methods and algorithms are directed toward supervised learning, which involves identifying patterns in existing data to make predictions about subsequently received data. Specifically, ML methods and algorithms directed toward supervised learning are “trained” through training data, which includes example inputs and associated example outputs. Based on the training data, the ML methods and algorithms may generate a predictive function that maps outputs to inputs and utilize the predictive function to generate ML outputs based on data inputs. The example inputs and example outputs of the training data may include any of the data inputs or ML outputs described above. For example, an ML module may receive training data comprising customer identification and geographic information and an associated customer category, generate a model that maps customer categories to customer identification and geographic information, and generate an ML output comprising a customer category for subsequently received data inputs including customer identification and geographic information.

In another aspect, ML methods and algorithms are directed toward unsupervised learning, which involves finding meaningful relationships in unorganized data. Unlike supervised learning, unsupervised learning does not involve user-initiated training based on example inputs with associated outputs. Rather, in unsupervised learning, unlabeled data, which may be any combination of data inputs and/or ML outputs as described above, is organized according to an algorithm-determined relationship. In one aspect, an ML module receives unlabeled data comprising customer purchase information, customer mobile device information, and customer geolocation information, and the ML module employs an unsupervised learning method such as “clustering” to identify patterns and organize the unlabeled data into meaningful groups. The newly-organized data may be used, for example, to extract further information about a customer's spending habits.

In yet another aspect, ML methods and algorithms are directed toward reinforcement learning, which involves optimizing outputs based on feedback from a reward signal. Specifically, ML methods and algorithms directed toward reinforcement learning may receive a user-defined reward signal definition, receive data input, utilize a decision-making model to generate an ML output based on the data input, receive a reward signal based on the reward signal definition and the ML output, and alter the decision-making model so as to receive a stronger reward signal for subsequently generated ML outputs. The reward signal definition may be based on any of the data inputs or ML outputs described above. In one aspect, an ML module implements reinforcement learning in a user recommendation application. The ML module may utilize a decision-making model to generate a ranked list of options based on user information received from the user and may further receive selection data based on a user selection of one of the ranked options. A reward signal may be generated based on comparing the selection data to the ranking of the selected option. The ML module may update the decision-making model such that subsequently generated rankings more accurately predict a user selection.

As will be appreciated based upon the foregoing specification, the above-described aspects of the disclosure may be implemented using computer programming or engineering techniques including computer software, firmware, hardware, or any combination or subset thereof. Any such resulting program, having computer-readable code means, may be embodied or provided within one or more computer-readable media, thereby making a computer program product, i.e., an article of manufacture, according to the discussed aspects of the disclosure. The computer-readable media may be, for example, but is not limited to, a fixed (hard) drive, diskette, optical disk, magnetic tape, semiconductor memory such as read-only memory (ROM), and/or any transmitting/receiving media, such as the Internet or other communication network or link. The article of manufacture containing the computer code may be made and/or used by executing the code directly from one medium, by copying the code from one medium to another medium, or by transmitting the code over a network.

These computer programs (also known as programs, software, software applications, “apps”, or code) include machine instructions for a programmable processor, and can be implemented in a high-level procedural and/or object-oriented programming language, and/or in assembly/machine language. As used herein, the terms “machine-readable medium” and “computer-readable medium” each refer to any computer program product, apparatus, and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The “machine-readable medium” and “computer-readable medium,” however, do not include transitory signals. The term “machine-readable signal” refers to any signal used to provide machine instructions and/or data to a programmable processor.

As used herein, a processor may include any programmable system including systems using micro-controllers, reduced instruction set circuits (RISC), application-specific integrated circuits (ASICs), logic circuits, and any other circuit or processor capable of executing the functions described herein. The above examples are examples only, and are thus not intended to limit in any way the definition and/or meaning of the term “processor.”

As used herein, the terms “software” and “firmware” are interchangeable and include any computer program stored in memory for execution by a processor, including RAM memory, ROM memory, EPROM memory, EEPROM memory, and non-volatile RAM (NVRAM) memory. The above memory types are examples only and are thus not limiting as to the types of memory usable for storage of a computer program.

In one aspect, a computer program is provided, and the program is embodied on a computer-readable medium. In one aspect, the system is executed on a single computer system, without requiring a connection to a server computer. In a further aspect, the system is being run in a Windows® environment (Windows is a registered trademark of Microsoft Corporation, Redmond, Washington). In yet another aspect, the system is run on a mainframe environment and a UNIX® server environment (UNIX is a registered trademark of X/Open Company Limited located in Reading, Berkshire, United Kingdom). The application is flexible and designed to run in various different environments without compromising any major functionality.

In some aspects, the system includes multiple components distributed among a plurality of computing devices. One or more components may be in the form of computer-executable instructions embodied in a computer-readable medium. The systems and processes are not limited to the specific aspects described herein. In addition, components of each system and each process can be practiced independently and separately from other components and processes described herein. Each component and process can also be used in combination with other assembly packages and processes. The present aspects may enhance the functionality and functioning of computers and/or computer systems.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Fragment-Count Method of Determining Cell State Composition From Serum CFDNA

This example describes a method for determining a cell state composition from a biological sample using the fragment-count method described herein. This example also discloses predictions of a therapeutic response and immune-related adverse event characteristics based on the cell state composition obtained using the disclosed systems and methods.

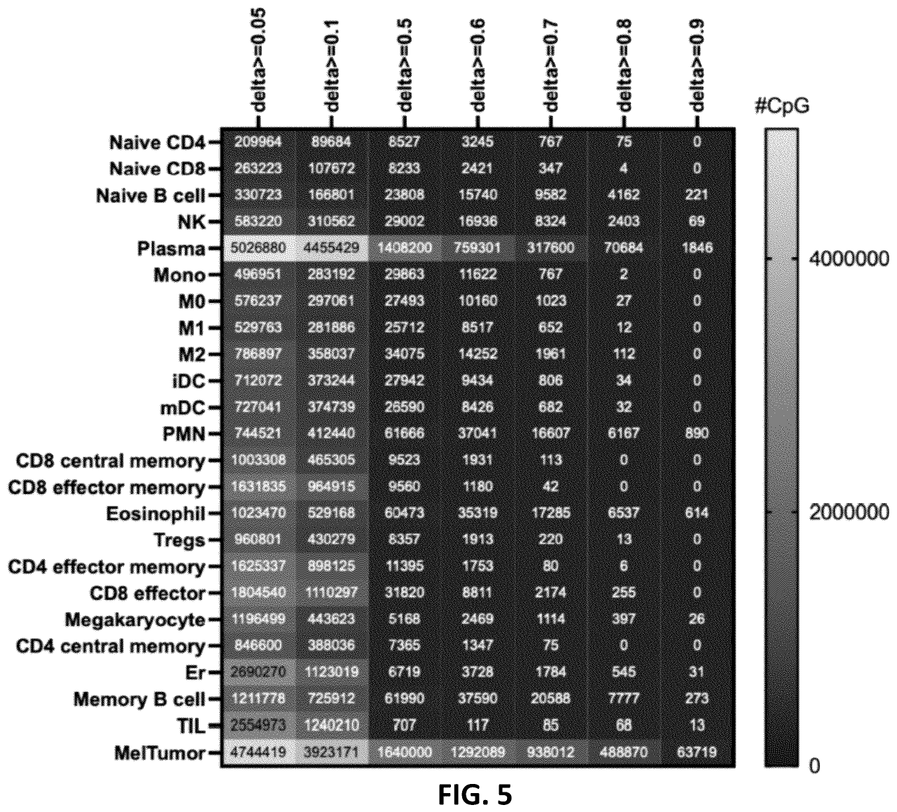

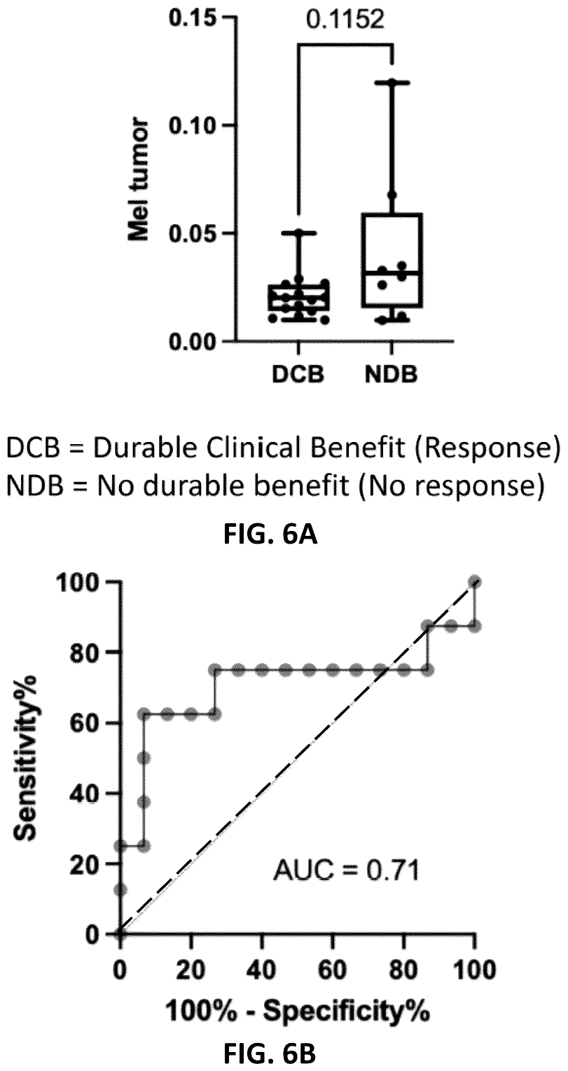

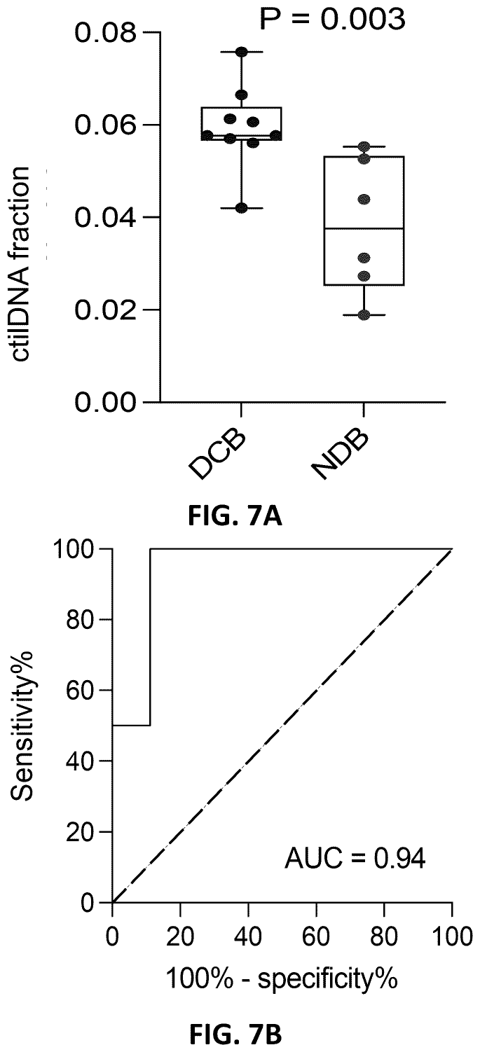

An example bin plot generated by the read-counting method of the present disclosure, showing the 24 cell types/states identified with the method, can be found in FIG. 5. To determine whether the read-counting method can predict immunotherapy response in melanoma patients, melanoma tumor DNA fraction (FIG. 6A) and tumor-infiltrating leukocyte (TIL) DNA fraction (FIG. 7A) in cell-free DNA samples from plasma from melanoma patients with or without durable clinical benefit were compared, and the sensitivity and specificity of the ability of the method to predict response was characterized (FIG. 6B and 7B), showing its ability to predict response.

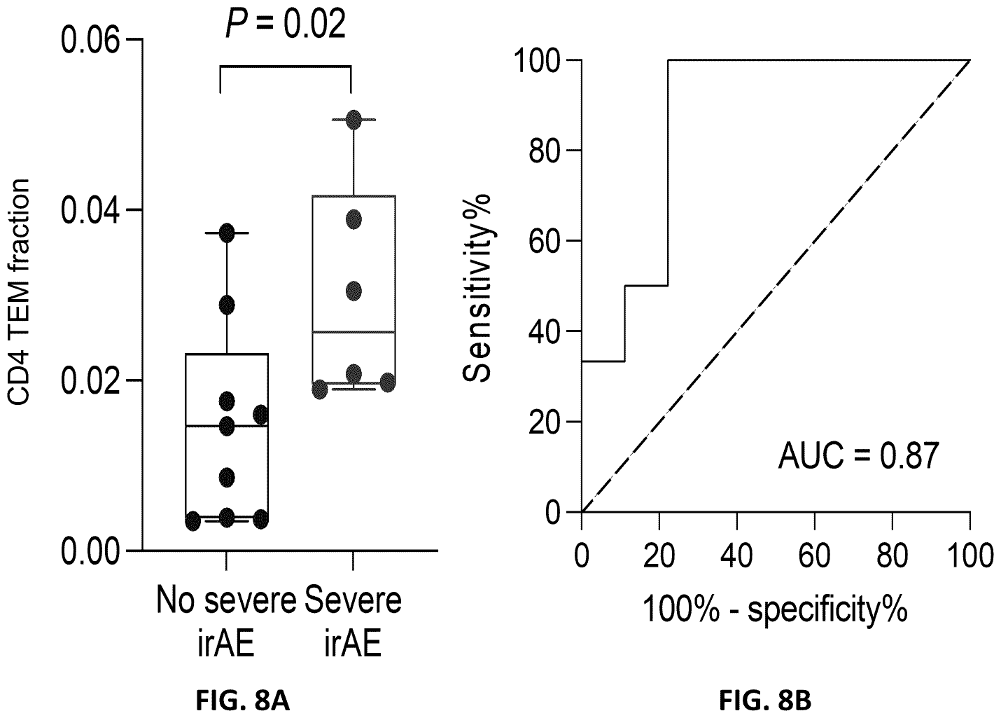

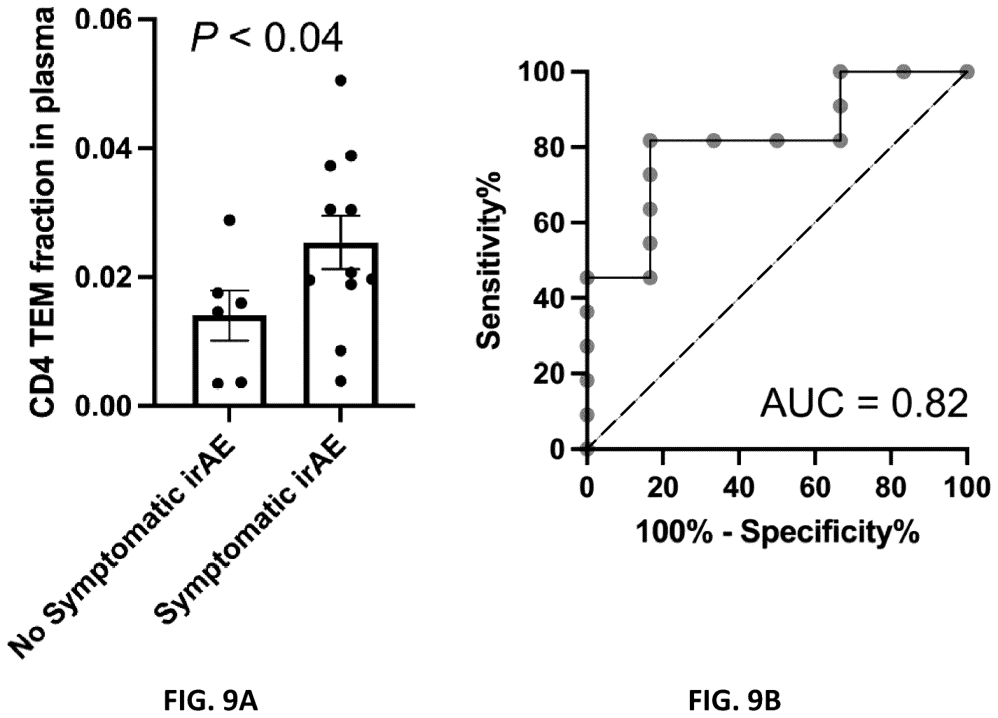

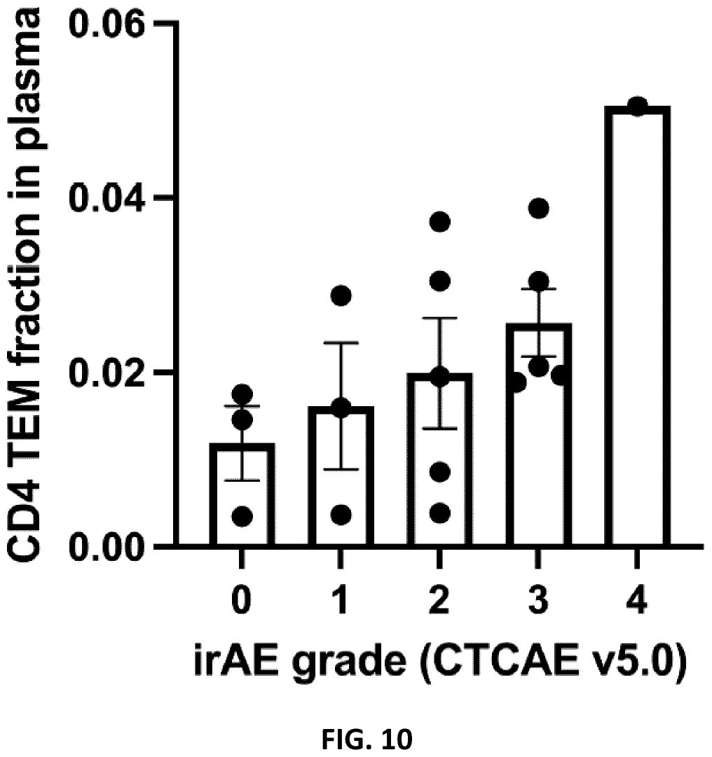

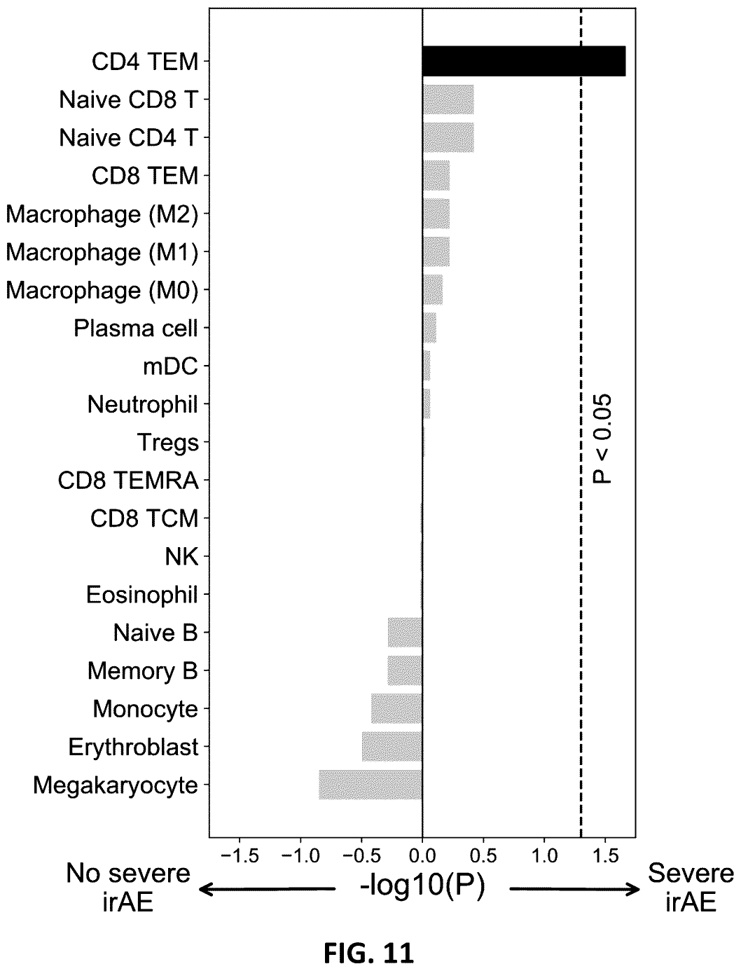

To determine whether the read-counting method can predict severe or symptomatic immune-related adverse effects (irAE) from immunotherapy in melanoma patients, CD4 TEM DNA fraction in cell-free DNA samples from plasma from melanoma patients with severe (FIG. 8A) or symptomatic (FIG. 9A) irAE were compared to patients without severe or symptomatic irAE. The sensitivity and specificity of the method's ability to predict severe (FIG. 8B) or symptomatic (FIG. 9B) irAE from immunotherapy was characterized, which showed that the method was able to predict severe and symptomatic irAE using the cell-free CD4 TEM fraction in the cell-free DNA samples. It is to be noted that cell-free CD4 TEM fraction was most significantly associated with severe irAE (FIG. 11). The method was also able to predict irAE on a grade-by-grade basis using the CD4 cell-free DNA (FIG. 10).

What is claimed is:

1. A method of determining a cell state composition from a biological sample, the method comprising:
(a) providing the biological sample comprising cell-free DNA, the cell-free DNA comprising a plurality of cell-free DNA fragments;
(b) providing a ground-truth reference table comprising a plurality of reference cell and tissue states and associated reference methylation levels;
(c) identifying CpGs within each DNA fragment of the cell-free DNA to determine a methylation level associated with each DNA fragment;
(d) comparing the methylation levels of each DNA fragment with the reference methylation levels associated with each cell and tissue state in the ground-truth reference table;
(e) assigning each DNA fragment to the cell or tissue state from the ground-truth reference table with an associated reference methylation level that is most similar to the methylation level of the DNA fragment;
(f) counting the numbers of DNA fragments assigned to each cell or tissue state of the ground-truth reference table to produce a read-count table; and
(g) determining the cell state composition based on the read-count table.

2. The method of claim 1, wherein the biological sample is a blood sample.

3. The method of claim 1, wherein the reference methylation values comprise differentially methylated CpGs derived from DNA originating from known cell types and known cell states, optionally of bacterial, viral, fungal, or eukaryotic parasitic origin.

4. The method of claim 1, wherein the cell-free DNA is plasma-derived.

5. The method of claim 1, wherein the cell state composition comprises at least two cell types, each cell type comprising at least two cell states.

6. The method of claim 1, further comprising inferring a melanoma tumor fraction, a tumor-infiltrating leucocyte fraction, a CD4 TEM level, and any combination thereof based on the cell state composition.

7. A method of predicting a therapeutic response of a subject to be administered an immunotherapy treatment, comprising:
(a) obtaining a biological sample from the subject comprising cell-free DNA, the cell-free DNA comprising a plurality of cell-free DNA fragments;
(b) determining the cell state composition of the subject by:
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a melanoma tumor fraction based on the cell state composition; and
(d) predicting the response to the immunotherapy treatment based on the melanoma tumor fraction.

8. A method of predicting a therapeutic response of a subject to be administered an immunotherapy treatment, comprising:
(a) obtaining a biological sample from the subject comprising cell-free DNA, the cell-free DNA comprising a plurality of cell-free DNA fragments;
(b) determining the cell state composition of the subject by:
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a tumor-infiltrating leucocyte fraction based on the cell state composition; and
(d) predicting the response to the immunotherapy treatment based on the tumor-infiltrating leukocyte fraction.

9. A method of predicting a severity of an immune-related adverse event of a subject to be administered an immunotherapy treatment, comprising:
(a) obtaining a biological sample from the subject comprising cell-free DNA, the cell-free DNA comprising a plurality of cell-free DNA fragments;
(b) determining the cell state composition of the subject by:
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a CD4 TEM fraction based on the cell state composition; and
(d) predicting the severity of an immune-related adverse event based on the CD4 TEM fraction.

10. A method of predicting a symptomatic immune-related adverse event of a subject to be administered an immunotherapy treatment, comprising:
(a) obtaining a biological sample from the subject comprising cell-free DNA, the cell-free DNA comprising a plurality of cell-free DNA fragments;
(b) determining the cell state composition of the subject by:
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a CD4 TEM fraction based on the cell state composition; and
(d) predicting the symptomatic immune-related adverse event based on the CD4 TEM fraction.

11. A method of predicting a grade of an immune-related adverse event of a subject to be administered an immunotherapy treatment, the method comprising:
(a) obtaining a biological sample from the subject comprising cell-free DNA, the cell-free DNA comprising a plurality of cell-free DNA fragments;
(b) determining the cell state composition of the subject by
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a CD4 TEM fraction, based on the cell state composition; and
(d) predicting the grade of the immune-related adverse event based on the CD4 TEM fraction.

12. A method of predicting a therapeutic response, a severe immune-related adverse event (irAE), a symptomatic irAE, an irAE grade, and any combination thereof of a subject to be administered an immunotherapy treatment, the method comprising:
(a) obtaining a single biological sample from the subject comprising cell-free DNA, the cell-free DNA comprising a plurality of cell-free DNA fragments;
(b) determining the cell state composition of the subject by:
(i) providing the biological sample comprising cell-free DNA, the cell-free DNA comprising a plurality of cell-free DNA fragments;
(ii) providing a ground-truth reference table comprising a plurality of reference cell and tissue states and associated reference methylation levels;
(iii) identifying CpGs within each DNA fragment of the cell-free DNA to determine a methylation level associated with each DNA fragment;
(iv) comparing the methylation levels of each DNA fragment with the reference methylation levels associated with each cell and tissue state in the ground-truth reference table;
(v) assigning each DNA fragment to the cell or tissue state from the ground-truth reference table with an associated reference methylation level that is most similar to the methylation level of the DNA fragment;
(vi) counting the numbers of DNA fragments assigned to each cell or tissue state of the ground-truth reference table to produce a read-count table; and
(vii) determining the cell state composition based on the read-count table;
(c) inferring a melanoma tumor fraction, a tumor-infiltrating leucocyte fraction, and a CD4 TEM fraction based on the cell state composition; and
(d) predicting at least one of:
(i) the response to the immunotherapy treatment based on at least one of the melanoma tumor fraction and the tumor-infiltrating leucocyte fraction; and
(ii) the severe immune-related adverse event (irAE), the symptomatic irAE, the irAE grade, and any combination thereof based on the CD4 TEM fraction.
